(19) United States
(12) Patent Application Publication
(10) Pub. No.: US 2002/0157163 A1
(43) Pub. Date: Oct. 31, 2002

(54) Transparent comfort device

(76) Inventor: David Chen, Walnut, CA (US)

Correspondence Address: CHRISTIE, PARKER & HALE, LLP, 350 WEST COLORADO BOULEVARD, SUITE 500, PASADENA, CA, 91105, US

(21) Appl. No.: 10/096,224

(22) Filed: Mar. 12, 2002

Related U.S. Application Data
(60) Provisional application No. 60/278,229, filed on Mar. 23, 2001.

(51) Int. Cl. A41D 13/08; A61F 9/00

(52) U.S. Cl. 2/16

(57) ABSTRACT

A comfort pad having a bladder formed from welded together thermoplastic sheets. At least one of the sheets are formed from a transparent material. There is a volume of transparent deformable gel material disposed within the bladder, and an integral pocket disposed along one of the thermoplastic sheets for accommodating a printed object for viewing through the gel material from a position above the comfort pad.

### FIELD OF THE INVENTION

This invention relates to a pad that can be interposed between a portion of a person's hand and a hard surface and, more specifically, to a pad that is placed between a person's hand and a hard surface to provide a comfortable resting place during operation of a hand-controlled device, such as a computer keyboard, mouse and/or trackball.

### BACKGROUND OF THE INVENTION

Typically, a computer user will use their hands for both inputting information into the computer via a keyboard, and for controlling the operation of the computer via a mouse or trackball. In an effort to improve a person's comfort in performing such hand-assisted computer operations, different types of comfort devices have been devised for placement between a person's hand and the adjacent hard surface. For example, rubber pads have been devised for placement on or near a computer keyboard to interface with a portion of a person's palm during keyboard input. Similarly, rubber pads have also been devised for placement near a mouse or trackball to interface with a portion of a person's palm during mouse or trackball operation. Configured and positioned in this manner, such devices serve to insulate a person's hand from contact with an adjacent hard surface. Instead, the person's hand is placed into contact with a deformable and soft piece of material that reduces the amount of contact pressure, and thus perceived discomfort, associated with performing the hand-assisted task. In an effort to even further increase one's comfort, such pads are known to be formed from a deformable gel rather than rubber.

Such comfort pads known in the art, formed from a gel material, are configured having an outside covering that is formed from a flexible material, such as spandex or the like. Configured in this manner, the pads are made having a number of differently colored covers. A disadvantage of using such conventional comfort pads is the working area or space around a computer is usually minimal depending on the specific work station. Thus, the decision to use such a comfort pad is usually made at the expense of giving up otherwise useful and needed space.

It is, therefore, desired that a comfort pad be configured in such a manner as to minimize or reduce the downside associated with losing desired work space.

### SUMMARY OF THE INVENTION

The present invention provides a comfort pad having a transparent or clear/see through outer cover that is filled with, and that contains, a deformable gel. The comfort pad is sized and shaped to facilitate placement between a person's hand and a hard surface, and includes an integral jacket for accommodating placement of a desired printed object therein for viewing from a position above the comfort pad.

The comfort pad comprises a bladder formed from welded together thermoplastic sheets. At least one of the sheets are formed from a transparent material. There is a volume of transparent deformable gel material disposed within the bladder, and an integral pocket is disposed along one of the thermoplastic sheets for accommodating a printed object for viewing through the gel from a position above the comfort pad.

The present comfort pad can also be constructed having a deformable member connected to the bladder for bending and removably attaching the comfort pad around a portion of a person's hand. Further, the comfort pad can be configured for use with a computer keyboard, where the comfort pad is positioned lengthwise across the front edge of the keyboard.

### BRIEF DESCRIPTION OF THE DRAWINGS

These and other features and advantages of the present invention will become appreciated as the same becomes better understood with reference to the specification, claims and drawings wherein:

FIG. 1 is a perspective top view of a comfort pad constructed according to principles of this invention;

FIG. 2 is a perspective bottom view of the comfort pad of FIG. 1, constructed according to principles of this invention;

FIG. 3 is a perspective view of an alternative embodiment of a comfort pad constructed according to principles of this invention; and

FIG. 4 is a perspective view of a still other embodiment of a comfort pad constructed according to principles of this invention.

### DESCRIPTION OF THE INVENTION

Comfort pads, constructed according to principles of this invention, generally comprise a transparent or clear/see through outer cover that is filled with, and that contains, a deformable gel. The comfort pad is sized and shaped to facilitate placement between a person's hand and a hard surface, and includes an integral jacket for accommodating placement of a desired printed object therein for viewing from a position above the comfort pad.

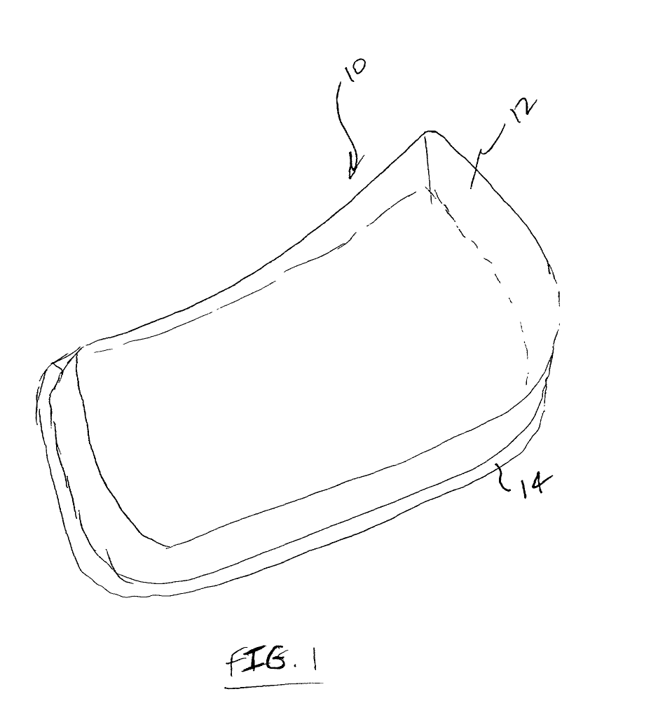

FIG. 1 illustrates a comfort pad 10 comprising a transparent or clear outer cover 12 that encloses a volume of a deformable gel material therein. Configured in this manner, the cover serves the function of a bladder for the volume of desired gel material. In an example embodiment, the outer cover is formed from a transparent polymeric material and is filled with a desired gel in a single thermo-forming process. The cover can be formed from any kind of thermoplastic film, such as polyurethane, polyethylene, polycarbonate, or polypropylene, of various thickness and softnesses depending on particular application. An exemplary thermoplastic film is polyurethane having a thickness in the range of from about 1 to 10 mils.

In an example embodiment, the comfort pad outer cover is formed by welding/sealing together two thermoplastic films, forming a gel bladder therebetween, using a manufacturing technique such as that described in U.S. Pat. No. 5,693,164. Other manufacturing techniques which can obtain a good welded seal 14 of the outer cover and/or between layers of thermoplastic film may also be employed. All that is needed for constructing a comfort pad is that the films be capable of being welded/sealed for enclosing the gel material therebetween, and that the films be substantially impervious to the gel material. Preferably, the film is selected so that the gel bonds to the film upon curing of the gel. Polyurethane film is preferred since it is among the toughest thermoplastic films available and is best able to stand the wear and abuse a person may give the comfort pad. Polyurethane film also bonds readily to the preferred silicone gels.

Although a silicone gel is preferred for forming comfort pads of this invention, other gel materials may be used such as hydro gel, PVC gel, or polyurethane gel. In some embodiments, these gel materials may be preferred since they are less costly than silicone gel. It is desired that the gel have a softness of between Shore 000 scale 0 and 40.

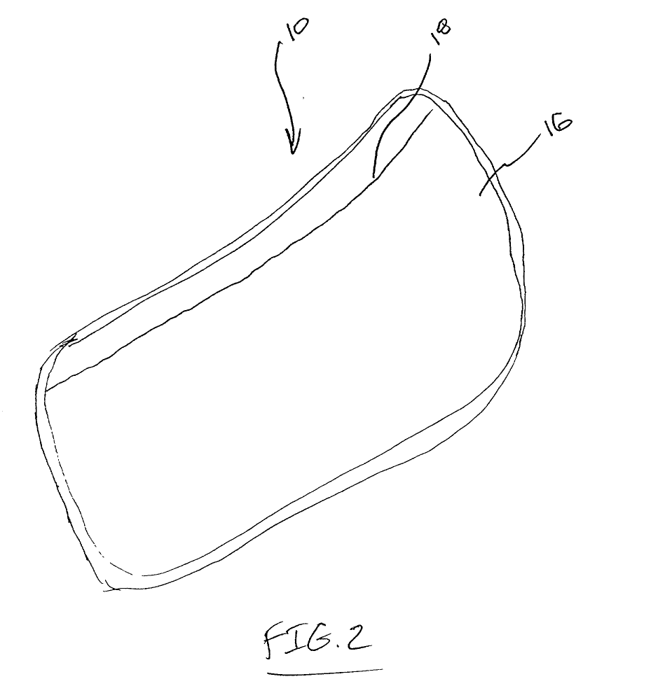

The comfort pad illustrated in FIG. 1 is one that is specifically configured to accommodate use with a person's hand during operation of a hand-assisted operation such as during operation of a computer mouse or a trackball. Referring to FIG. 2, a key feature of the comfort pad 10 is the placement of an integral pocket 18 along a backside surface 16 of the outer cover. The pocket 18 is sized and configured to accommodate placement of a flat object therein. In an example embodiment, the flat object could be in the form of a printed object that is slid and contained within the pocket. The printed object could be in the form of a photograph, drawing or written object.

Since the comfort pad cover and gel are both transparent or clear, the printed object positioned within the pocket is readily viewable from a position above the comfort pad. This feature is important as it allows a person to view the printed object from their work position, thereby eliminating any space consuming detriment associated with the comfort pad, i.e., the comfort pad could occupy that region of space previously occupied by the printed object without sacrificing the ability of being able to view the printed object. Further, it allows the comfort pad to serve as a decorative item that can be customized by each user, and changed by inserting a different flat object within the integral pocket whenever a different appearance is desired.

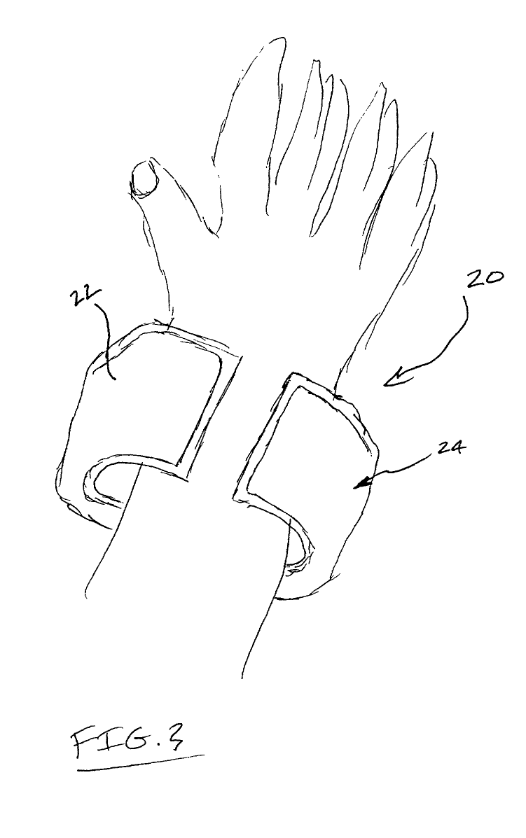

FIG. 3 illustrates an alternative comfort pad 20 of this invention that is configured similar to the comfort pad 10 described above, but further configured to accommodate removable placement of the pad around a person's wrist or portion of the hand. In such embodiment, the comfort pad 20 is configured having a deformable member 22 attached to or embedded along a backside surface of the outer cover. The deformable member 22 can be in the form of metal wires or a metal plate that is either permanently attached to the backside surface, e.g., serving as a cover base plate, or that can be inserted in an integral pocket formed within the backside surface. Alternatively, the deformable member 22 can be inserted between the thermoplastic sheets, wherein the deformable member is encased within the bladder and surrounded directly by the gel material. Moreover, the deformable member can be made from a deformable plastic material, preferably a clear plastic material, which would allow the deformable member to be transparent.

Since the comfort pad is formed from a cover and gel material that is clear or transparent, the deformable member can be configured having a flat object, e.g., a printed object such as a photograph or picture, disposed thereon for viewing from outside of the comfort pad. Alternatively, the deformable member can be used in conjunction with a separate printed object that is positioned within the integral pocket for its viewing from outside of the comfort pad. Furthermore, the deformable member can have an image etched directly into its surface, or have a decorative shape or configuration. For example, a company logo or other symbol can be etched, engraved, or molded onto the deformable member and would be readily viewed through the outer cover and gel material of the comfort pad.

A key feature of this particular comfort pad embodiment is the use of the deformable member that enables the comfort pad to be bent or otherwise removably deformed around a person's wrist or hand, thereby enabling movement of the comfort pad with the person's hand during operation of a hand-assisted task, e.g., during the movement of a computer mouse or trackball. Thus, unlike the comfort pad embodiment discussed above and illustrated in FIGS. 1 and 2, use of this embodiment comfort pad provides the user with a greater degree of comfortable hand movement.

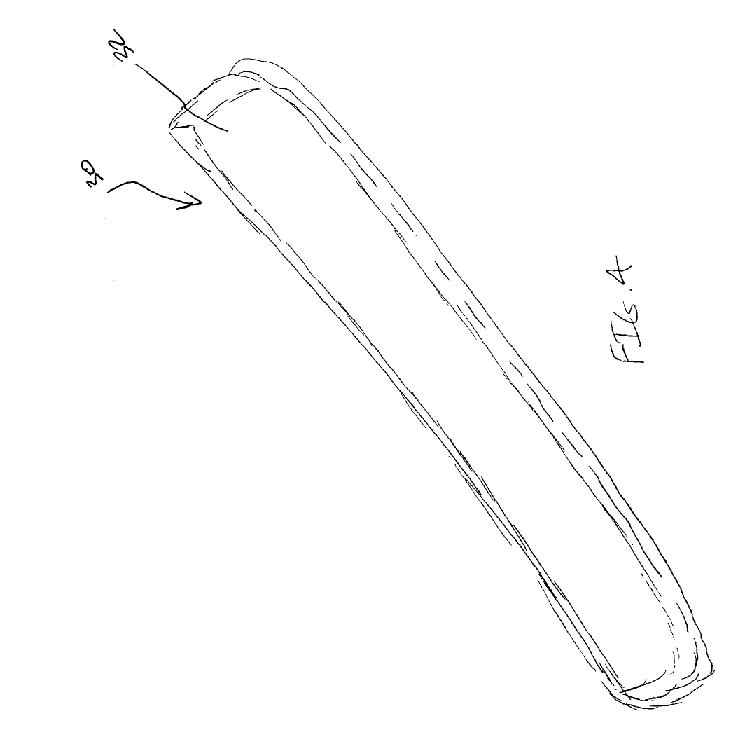

FIG. 4 illustrates a still other embodiment of the comfort pad 30 of this invention as configured to be used with a computer keyboard. The main difference between this invention embodiment and that discussed and illustrated above in FIGS. 2 and 3, is the shape of the pad. Specifically, the comfort pad 30 of this embodiment is shaped to permit its placement lengthwise across the leading or front edge of a keyboard to make contact with both palms of a person's hand. Like the embodiments described above, this comfort pad embodiment too comprises a transparent or clear outer cover 32, formed from a pair of joined thermoplastic films, that contains a desired volume of silicone gel therein. The cover preferably includes a backside surface having an integral jacket therein for containing a desired printed object for viewing from a position above the comfort pad.

These are but a few examples of embodiments of comfort pads constructed according to principles of the invention to provide an improved degree of interface comfort afforded by a person using the pad to perform certain hand-assisted tasks. It is to be understood that the comfort pads of this invention can be configured in any shape and in any size to accommodate different types of applications, which can include applications other than those specifically described above. Accordingly, it is to be understood that, within the scope of the appended claims, comfort pads constructed according to principles of this invention may be embodied other than as specifically described herein to address needs of particular applications other than those described herein.

What is claimed is:

1. A comfort pad comprising:
a bladder formed from welded together thermoplastic sheets, wherein at least one of the sheets are formed from a transparent material;
a volume of transparent deformable gel material disposed within the bladder; and
an integral pocket disposed along one of the thermoplastic sheets for accommodating a flat object therein for viewing through the gel material from a position above the comfort pad.

2. The comfort pad as recited in claim 1 wherein the thermoplastic sheets are made from polyurethane.

3. The comfort pad as recited in claim 1 wherein the gel material is a silicone gel.

4. The comfort pad as recited in claim 1 further comprising a deformable member adjoined with the bladder for bending and removably attaching the comfort pad around a portion of a person's hand.

5. The comfort pad as recited in claim 4 wherein the deformable member is made from a metal.

6. The comfort pad as recited in claim 4 wherein the deformable member is made from a clear plastic material.

7. The comfort pad as recited in claim 4 wherein the deformable member has an image positioned on at least one outer surface for viewing through the gel material from the position above the comfort pad.

8. The comfort pad as recited in claim 1 wherein the comfort pad is configured for placement lengthwise across a front edge of a computer keyboard.

9. The comfort pad as recited in claim 1 wherein the flat object is a printed object.

10. A comfort pad comprising:
a bladder formed from sealed together thermoplastic sheets, wherein at least one of the sheets are formed from a transparent material;
a volume of transparent deformable gel material disposed within the bladder; and
a deformable member adjoined with the bladder for bending and removably attaching the comfort pad around a portion of a persons hand.

11. The comfort pad as recited in claim 10 wherein the deformable member is disposed within a separate pocket that is adjoined to the bladder.

12. The comfort pad as recited in claim 10 wherein the deformable member is disposed within the bladder.

13. The comfort pad as recited in claim 10 further comprising an integral pocket disposed along one of the thermoplastic sheets for accommodating a flat object therein for viewing through the gel material from a position above the comfort pad.

14. The comfort pad as recited in claim 10 wherein the thermoplastic sheets are made from polyurethane.

15. The comfort pad as recited in claim 10 wherein the gel material is a silicone gel.

16. The comfort pad as recited in claim 10 wherein the deformable member is made from a metal.

17. The comfort pad as recited in claim 10 wherein the deformable member is made from a clear plastic material.

18. The comfort pad as recited in claim 10 wherein the deformable member has an image positioned on at least one outer surface for viewing through the gel material from a position above the comfort pad.
